(19) United States
(12) Patent Application Publication
(10) Pub. No.: US 2001/0044610 A1
(43) Pub. Date: Nov. 22, 2001

(54) Absorbent article with fluid intake intensifier

(76) Inventors: Hyung Byum Kim, DaeJon (KR); Eun Jung Kang, Seoul (KR); Eo Yeon Hwang, AnYang-City (KR); Doo Hong Kim, Nonthaburi (TH)

Correspondence Address: Thomas M. Parker,, Kimberly-Clark Worldwide, Inc., Patent Department, 401 North Lake Street, Neenah, WI, 54956, US

(21) Appl. No.: 09/750,744

(22) Filed: Dec. 28, 2000

Related U.S. Application Data
(60) Provisional application No. 60/173,912, filed on Dec. 29, 1999.

(51) Int. Cl. A61F 13/15; A61F 13/20

(52) U.S. Cl. 604/365; 604/366; 604/367; 604/378

(57) ABSTRACT

Disposable absorbent articles having a fluid intake pledget. Disposable absorbent articles such as feminine hygiene products (e.g., sanitary napkins and panti-liners) and incontinence products having an airlaid, lofty pledget of a low density, nonwoven material for intake of body fluids.

### BACKGROUND

The present invention relates to disposable absorbent articles having an enhanced fluid intake intensifier pledget. More particularly, the present invention relates to disposable absorbent articles having a low density, non-woven fluid intake intensifier pledget.

Absorbent articles such as sanitary napkins and incontinence pads are well known in the personal care and hygiene art. Such absorbent articles are generally constructed with a fluid pervious cover, a liquid impervious baffle, and an absorbent core positioned between the cover and the baffle. The fluid pervious cover generally allows body fluids such as, for example, urine and/or menses, to seep through to the absorbent core which is typically designed to retain body fluids while the liquid impervious baffle prevents further seepage.

Problems which have typically existed in the art are the ability of the fluid pervious cover to allow quick seepage (i.e., intake) in one direction towards the conventional absorbent core while preventing return in the opposite direction (i.e., rewet). The rate at which intake occurs generally determines whether side leakage is reduced or whether body fluids are appropriately concentrated in a desired target area.

Historically, many solutions have been proposed. The planar design of many absorbent articles has been modified in a multitude of ways in an effort to supplement the above-described general depth structure. Absorbent articles have been designed to conform in shape to the crotch area so that concave side portions fit the natural curves of the thighs. The reduction of the lateral sides of the planar design in conventional absorbent articles often resulted in side leakage which soiled the undergarment and clothing of the wearer. To address such side leakage, lateral wings and flaps were introduced to certain absorbent articles making designs of such absorbent articles progressively more complicated.

Supplementing planar design of the absorbent articles were efforts to concentrate body fluids to confined areas. Embossed channels were stamped onto the body-facing surface of absorbent articles to define physical target areas where it was hoped that body fluid would concentrate, usually in central areas of the absorbent article. Several shaped designs for the embossed areas have been proposed ranging from the straight periphery, rectangular shape to variations such as curved or arcuate peripheries, hour-glass and race-track configurations.

Another direction attempted was the use of a surge layer. A surge layer is typically a strip made of porous non-woven material having a planar size usually smaller than that of the absorbent core and used as a composite with the fluid pervious cover. Variations to the use of a surge layer include composites with the entire planar surface of a fluid pervious cover. A surge layer is oftentimes laminated to the fluid pervious cover by thermal, ultrasonic or mechanical bonding, or combinations thereof to ensure its position in the target area. Thus, a surge layer may be laminated to the target area or laminated onto substantially the entire surface of the fluid pervious cover. Conventional surge materials have low porosity and resilience and deteriorate during bonding with the cover. Due to these drawbacks, the desirable advantages such as increased intake and reduced rewet are at times not achieved.

Another conventional approach is to use an absorbent material such as fiberized pulp in a double layer along the length of the absorbent core or concentrated in the central target area. However, this approach may provide discomfort to the wearer especially during activity or movement.

### SUMMARY

The present inventors have recognized the difficulties and problems inherent in the prior art and in response thereto conducted intensive research in developing an enhanced fluid intake intensifier pledget for disposable absorbent articles. These intensive research efforts resulted in various embodiments of the invention described herein. In one embodiment, the present invention is directed to an absorbent article having an absorbent core and a fluid intake intensifier pledget located on a central portion of the absorbent core. The pledget typically includes a Thru-Air Bonded Carded Web (TABCW) material, an airlaid material, a composite of a fiber material and an airlaid material, or combinations of a fiber layer and an airlaid layer. The combination of a fiber layer and an airlaid layer includes two distinct layers, a first layer and a second layer wherein the first layer includes the fiber material and the second layer includes the airlaid material.

The TABCW material generally provides a low densified lofty thru-air bonded carded web having a basis weight ranging from about 15 to about 75 grams per square meter (gsm). The TABCW material can include about 3 to about 10 denier staple fiber. The TABCW material can also include an Ultra Bulky (UB) bicomponent fiber or composites thereof. The airlaid nonwoven material has a basis weight ranging from about 50 to about 300 gsm and typically provides for high void volume. The airlaid nonwoven material may include a superabsorbent material.

The pledget may also include a first layer and a second layer, wherein the first layer includes the TABCW material and the second layer includes the airlaid nonwoven material. The pledget may further include a composite of the TABCW material and the airlaid nonwoven material.

The absorbent core may be a superabsorbent particulate (SAP) sheet which is a composite of a superabsorbent material and pulp, tissue, a nonwoven material or a mixture of fluff and superabsorbent material. The absorbent core may further include a tissue-like wrapping or nonwoven material.

The pledget located on the central portion of the absorbent core typically has a length of at least about 50 mm and a width ranging from about 30 to about 60 mm. The absorbent article can also include a fluid pervious cover, a wrapping material and a baffle portion. The pledget has a first surface situated adjacent the cover and a second surface adhesively bonded to at least one of the absorbent core or the wrapping material. The absorbent article can further include a fluid distribution layer. The cover, the wrapping material, the absorbent core, and the baffle may be compressed to a desired thickness in the Z-direction to form a channel around the pledget. The width of the embossed channel is generally less than about 1 cm. Typically, any such channel is situated approximately adjacent to the periphery of the pledget. The cover sheet is typically of a fluid pervious material which is adapted to allow body fluid to seep through to the pledget. The baffle is generally of a liquid impervious material adapted to prevent further seepage of body fluids.

In another embodiment of the absorbent article of the present invention, a combination of a cover including a hydroentangled, hydroapertured spun-lace material and a pledget comprised of a TABCW material is synergistically used to speed up intake time, reduce rewet and enhance fluid retention by the absorbent core. The hydroentangled, hydroapertured spun-lace material can comprise a rayon fiber, and desirably a homogeneous mixture of about 70% rayon fiber and about 30% polyethylene terephthalate (PET) polyester fiber. The hydroentangled, hydroapertured spun-lace material can also comprise PET polyester, polyethylene, polypropylene or bicomponents thereof. The term “bicomponent” as used herein is intended to refer to fiber mixtures of PET polyester, polyethylene and/or polypropylene as understood in the art.

A further embodiment of the absorbent article of the present invention includes a cover, a first absorbent layer and a second absorbent layer, wherein the first absorbent layer is situated between the cover and the second absorbent layer. In this embodiment, the cover may comprise a hydroentangled, hydroapertured spun-lace material as described herein. The first absorbent layer includes a material selected from the group consisting of an airlaid material, a TABCW material and a composite of a fiber material and an airlaid material. The second absorbent layer includes a material selected from the group consisting of an airlaid material, a TABCW material and a composite of a fiber material and an airlaid material.

### DRAWINGS

The foregoing and other features, aspects and advantages of the present invention will become better understood with regard to the following description, appended claims and accompanying drawings where:

FIG. 1 illustrates a plan view of an absorbent article having an embodiment of a fluid intake intensifier pledget.

FIG. 2 illustrates an enlarged, cross-sectional view of the absorbent article of FIG. 1 taken along line 2-2 of FIG. 1.

FIG. 3 illustrates a plan view of an absorbent article having an alternate embodiment of a fluid intake intensifier pledget.

FIG. 4 illustrates an enlarged, cross-sectional view of the absorbent article of FIG. 3 taken along line 4-4 of FIG. 3.

FIG. 5 illustrates a plan view of an absorbent article having another embodiment of a fluid intake intensifier pledget.

FIG. 6 illustrates an enlarged, cross-sectional view of the absorbent article of FIG. 5 taken along line 6-6 of FIG. 5.

### DESCRIPTION

The present invention is directed generally to disposable absorbent articles having an enhanced fluid intake intensifier pledget. More particularly, this invention is directed to disposable absorbent articles having a low density, non-woven fluid intake intensifier pledget. The fluid intake intensifier pledget can include a Thru-Air Bonded Carded Web (TABCW) material, an airlaid nonwoven material, a composite of fiber material and airlaid material, or combinations of a fiber layer and an airlaid layer. The combination of a fiber layer and an airlaid layer has two distinct layers, a first layer and a second layer wherein the first layer includes the fiber material and the second layer includes the airlaid material.

As used herein, the phrase “absorbent article” refers to devices which absorb and contain body fluids, and more specifically, refers to devices which are placed against or near the skin to absorb and contain the various fluids discharged from the body. The term “disposable” is used herein to describe absorbent articles that are not intended to be laundered or otherwise restored or reused as an absorbent article after a single use. Examples of such disposable absorbent articles include, but are not limited to, personal care absorbent products such as feminine hygiene products (e.g., sanitary napkins and panti-liners), diapers, training pants, incontinence products, and the like.

Disposable absorbent articles, such as, for example, many of the personal care absorbent products, typically comprise a fluid pervious cover, a liquid impervious baffle joined to the cover and an absorbent core positioned between the cover and the baffle. Disposable absorbent articles and components thereof, including the cover, baffle, absorbent core and any individual layers of these components, have a body-facing surface and a garment-facing surface. As used herein, “body-facing surface” means that surface of the article or component which is intended to be worn toward or placed adjacent to the body of the wearer, while the “garment-facing surface” is on the opposite side and is intended to be worn toward or placed adjacent to the wearer's undergarment when the disposable absorbent article is worn.

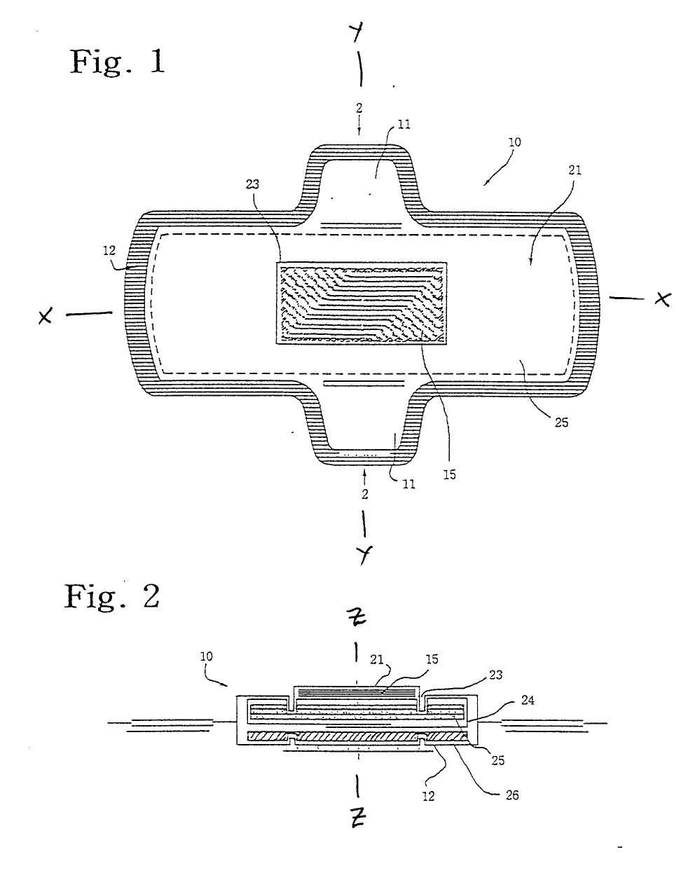

Referring to FIG. 1, a first embodiment of the enhanced fluid intake intensifier pledget of the present invention will now be described. For discussion purposes, the disposable absorbent article will be described with reference to a sanitary napkin. FIG. 1 illustrates a plan view of a sanitary napkin 10 having a pair of wings 11, a baffle 12, a fluid intake intensifier pledget 15, a cover 21, an optional embossed channel 23 and an absorbent core 25. The baffle 12 is shown extending along the entire periphery of the sanitary napkin 10. The cover 21 is illustrated as being substantially transparent and overlies both the fluid intake intensifier pledget 15 and the absorbent core 25. The wings 11 can be considered lateral extensions of the baffle 12, the cover 21 or both. Although illustrated as having wings, one skilled in the art will readily appreciate that the present invention can be incorporated into a sanitary napkin that does not include wings.

The cover 21 is designed to contact the body of the wearer and therefore should be easily penetrated by body fluids such as, for example, blood, menses and urine. The cover 21 should also be non-irritating to the wearer's skin and, desirably, will not absorb an appreciable amount of body fluid insulting its surface. The cover 21 can be constructed of a woven or nonwoven, natural or synthetic material. Suitable materials include bonded carded webs of polyester, polypropylene, polyethylene, nylon or other heat-bondable fibers. Other polyolefins, such as copolymers of polypropylene and polyethylene, linear low-density polyethylene, finely-perforated film webs and net material, also work well. Particularly desired are composite materials of a polymer and a nonwoven fabric material. Still another cover material is a spunbond web of polypropylene. The web can also contain about 1% to about 6% titanium dioxide pigment to give it a clean, white appearance. A uniform spunbond material is desirable because it has sufficient strength in the longitudinal direction, even after being perforated, to resist being torn or pulled apart during use. The most desired polypropylene webs have a basis weight of between about 18 and about 40 grams per square meter (gsm). Alternatively, the cover 21 includes a transparent film, a non-woven material or a laminate of a film and a non-woven material. The cover 21 can also include a spun-lace material. For example, the spun-lace material can include a rayon fiber and, desirably, a homogeneous mixture of about 70% rayon fiber and about 30% polyethylene terephthalate (PET) fiber. It should be noted, however, that cotton fiber can be used in place of rayon fiber. The ratio of rayon fiber or cotton fiber to PET fiber may vary from about 10:0 to about 0:10 and the average basis weight can range from about 20 to about 40 gsm. The spun-lace material can also include PET, polyethylene, polypropylene or bicomponent combinations thereof. The cover can also be constructed of a thermoplastic film flanked on both sides by a nonwoven material. This particular embodiment provides a soft feel against the wearer's thighs.

In order to facilitate movement of body fluid down into the sanitary napkin 10, a plurality of apertures may be formed in the cover 21. The apertures can be randomly or uniformly arranged throughout the cover 21, or they can be located only in a narrow longitudinal band or strip arranged substantially along the longitudinal axis X-X of the sanitary napkin 10. The apertures permit rapid penetration of body fluid down into the sanitary napkin 10. The size, shape, diameter and number of apertures can vary to suit one's particular needs. Desirably, the apertures extend over at least about 20% of the total surface area of the cover 21.

To aid in penetration of body fluid, the cover 21 can also be treated with a surfactant to improve its hydrophilic characteristics. The surfactant can include topical additions or internally applied materials like polysiloxanes.

The liquid impermeable baffle 12 is designed to permit the passage of air or vapor out of the sanitary napkin 10 while blocking the passage of liquids. The baffle 12 can be made from any material having the above-identified properties. A good material is a micro-embossed, polymeric film such as polyethylene or polypropylene. Bi-component films can also be used. Desirably, the baffle 12 will be a polyethylene film having a thickness in the range of about 0.2 to about 2.0 mm, most desirably from about 0.3 to about 1.0 mm.

The cover 21 and the baffle 12 can be coextensive and in contact around the periphery of the absorbent core 25. The cover 12 and the baffle 12 can also be sealed together about their peripheries by use of an adhesive, by heat sealing, by ultrasonics, or by any other process known to those skilled in the art. For example, the cover 21, the baffle 12 and at least a portion of the absorbent core 25 can be adhesively joined together and then die cut to have a common periphery. Alternatively, the cover 21 and the baffle 12 can cooperate together to encircle or wrap the absorbent core 25.

As illustrated in FIG. 1, located on a central portion of the sanitary napkin 10 is a fluid intake intensifier pledget 15 surrounded by an optional embossed channel 23. Although FIG. 1 illustrates the fluid intake intensifier pledget 15 as having a rectangular shape, it will be apparent to one skilled in the art that other shapes and configurations such as, for example, square, curved, arcuate, racetrack or hourglass are also possible while remaining within the scope of the present invention. The fluid intake intensifier pledget 15 may include two layers, with each layer having the same shape or a different shape. As illustrated in FIG. 1, the fluid intake intensifier pledget 15 is substantially aligned along the longitudinal axis X-X of the sanitary napkin 10. To adequately cover the central portion of the sanitary napkin 10, it is desired that the fluid intake intensifier pledget 15 has a length of at least about 50 mm and a width or widths of between about 30 and about 60 mm.

The fluid intake intensifier pledget 15 desirably has a body-facing surface situated adjacent the garment-facing surface of the cover 21 and a garment-facing surface typically adjacent to at least one of the absorbent core 25 or the wrapping material 24. The fluid intake intensifier pledget 15 desirably includes an airlaid nonwoven material, a TABCW material or a composite of a fiber material and an airlaid material, or combinations of a fiber layer and an airlaid layer. It is desired to use a thermally-bonded airlaid adhesive bonding process to affix the airlaid material to the absorbent core 25. Alternatively, a binder-bonded or a multi-bonded airlaid material may be used to bond the material to the absorbent core 25. Desirably, the airlaid material has a basis weight of about 50 to about 300 gsm and provides for high void volume. The airlaid material may include superabsorbent material. For the TABCW material, it is desired to use a low density, lofty TABCW material having a basis weight of about 15 to about 70 gsm and including a staple fiber having a denier ranging from about 3 to about 6. Alternatively, the TABCW may include an Ultra Bulky (UB) bicomponent fiber. The staple fiber included in the various embodiments of the present invention may be any staple fiber that improves absorbency or other property of the fluid intake intensifier pledget. Suitable staple fibers include polyester fibers, nylon fibers, cotton fibers and wood (pulp) fibers. The desired staple fiber is a wood fiber as the wood fibers formed from pulp are of desired size, low in cost and of high absorbency.

The airlaid nonwoven material and the TABCW material typically have relatively higher void volume capacity for greater absorption than conventional surge materials or absorbent cores, making the present invention particularly suitable for use in thin sanitary napkins or panti-liners. In addition, airlaid materials and TABCW materials provide rapid absorption of body fluid thus minimizing unnecessary discomfort to the wearer of the sanitary napkin. The high void volume of the fluid intake intensifier pledget 15, in addition to rapid absorption, provides greater retention of body fluid to avoid rewet. A combination of a top or a first layer including a TABCW material and a bottom or a second layer of an airlaid material is desired to enhance functionality of the fluid intake intensifier pledget 15 of the present invention.

Unlike conventional methods of using double layers of absorbent core material or the surge layer laminated to the cover, the present invention introduces the use of airlaid nonwoven materials in the fluid intake intensifier pledget 15 to provide a more zoned, area-concentrated and intensified fluid intake which substantially reduces lateral run-off and leakage. The low density, nonwoven materials used also provide a more cushioned feel due to their resiliency while being less physically bulky.

FIG. 2 illustrates an enlarged cross-sectional view taken along line 2-2 of FIG. 1. Beginning from the body-facing surface of the cover 21, FIG. 2 illustrates a sanitary napkin 10 as comprising a cover 21, a fluid intake intensifier pledget 15, a wrapping material 24, an absorbent core 25 and a baffle 12. FIG. 2 also illustrates an optional embossed channel 23 and an optional fluid distribution layer 26. The fluid distribution layer 26 includes a nonwoven material of pulp fibers and facilitates movement of body fluids to areas of the absorbent core 25 distant from the central portion (located approximate the intersection of the X-X and Y-Y axes) of the sanitary napkin 10. The wrapping material 24 can include a tissue, an air-laid material or a non-woven material and functions to minimize potential migration of the superabsorbent material which may be present in the absorbent core 25. The absorbent core 25 can be a composite or a laminate which includes a hydrophilic material and a superabsorbent material. The absorbent core 25 typically includes a sheet material having either densified or undensified non-woven material, superabsorbent material, tissue material or pulp material, or combinations thereof. The wrapping material 24 can be in an e-fold or c-fold configuration. The embossed channel 23 may be formed by compressing portions of the cover 21, the absorbent core 25, the baffle 12 and any other components of the sanitary napkin situated between the cover 21 and the baffle 12 to a desired thickness in the Z-direction. The width of the embossed channel is typically less than about 1 cm.

In a further embodiment of the present invention illustrated in FIG. 3, corresponding similar parts are denoted by the same numerals as in FIGS. 1 and 2. FIG. 3 illustrates a plan view of a sanitary napkin 10 having a pair of wings 11, a baffle 12, a fluid intake intensifier pledget 15, a cover 21 and an absorbent core 25. The baffle 12 is shown extending along the entire periphery of the sanitary napkin 10. The cover 21 is shown as being substantially transparent and overlies both the fluid intake intensifier pledget 15 and the absorbent core 25. Although illustrated as having wings, one skilled in the art will readily appreciate that the present invention can be incorporated into a sanitary napkin that does not include wings. Located on a central portion (situated approximate the intersection of the X-X and Y-Y axes) of the sanitary napkin 10 is the fluid intake intensifier pledget 15. Although illustrated as being rectangular in shape, the fluid intake intensifier pledget 15 may also be any of a variety of other geometric shapes such as, for example, an hourglass configuration. FIG. 3 also illustrates that the fluid intake intensifier pledget 15 may have an overall length which extends essentially the entire length of the sanitary napkin 10. Consequently, it is within the scope of the present invention that the fluid intake intensifier pledget 15 may substantially cover the entire body-facing surface of the absorbent core 25.

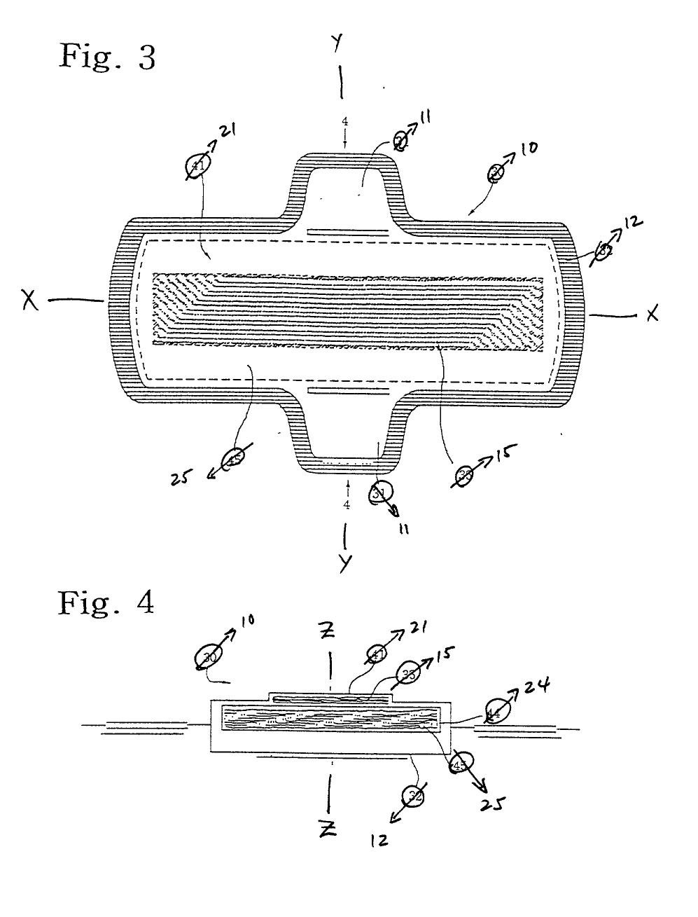

FIG. 4 illustrates an enlarged cross-sectional view taken along line 4-4 of FIG. 3. If desired, the layers shown in FIG. 4 can be compressed to a desired thickness in the Z-direction to form a channel around the intake intensifier pledget 15. The width of the embossed channel is typically less than about 1 cm. Beginning from the body-facing surface of the cover 21, FIG. 4 illustrates a sanitary napkin 10 as including a cover 21, a fluid intake intensifier pledget 15, a wrapping material 24, an absorbent core 25, and a baffle 12. Although not illustrated, the sanitary napkin 10 may also include a fluid distribution layer situated adjacent the body-facing surface of the baffle 12. The cover 21 and the baffle 12 are sealed at their edges to form a sealing margin with various patterns and using various processes as previously noted. The materials used for the fluid intake intensifier pledget 15 are substantially similar to those described for the embodiment illustrated in FIGS. 1 and 2.

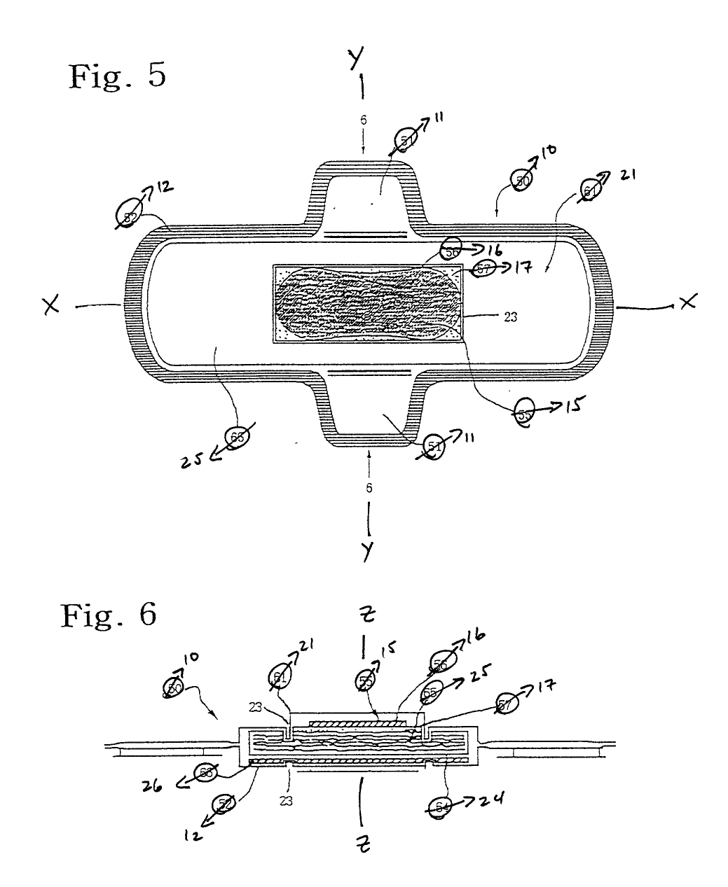

In yet another embodiment of the present invention illustrated in FIG. 5, corresponding similar parts are denoted by the same numerals as in FIGS. 3 and 4. FIG. 5 illustrates a plan view of a sanitary napkin 10 which includes a pair of wings 11, a baffle 12, a fluid intake intensifier pledget 15, a cover 21 and an absorbent core 25. The baffle 12 is shown extending along the entire periphery of the sanitary napkin 10. The cover 21 is illustrated as being substantially transparent. The fluid intake intensifier pledget 15 is substantially aligned along the longitudinal axis X-X of the sanitary napkin 15. For desired coverage of the central portion of the sanitary napkin 10, the fluid intake intensifier pledget 15 may be in the shape of an hourglass as illustrated. However, various other shapes and configurations such as, for example, rectangular and racetrack are also contemplated as being within the scope of the present invention.

FIG. 6 illustrates a cross-sectional view taken from line 6-6 of FIG. 5. FIG. 6 illustrates a sanitary napkin 10 having a cover 21, a fluid intake intensifier pledget 15, a wrapping material 24, an absorbent core 25 and a baffle 12. The materials used for the fluid intake intensifier pledget 15 and the other components of the sanitary napkin 10 are substantially similar to those disclosed for the previously described embodiments. The absorbent core 25 can be a composite or a laminate which includes a hydrophilic material and, optionally, a superabsorbent material. The absorbent core 25 may have two layers, a top or a first layer including unwrapped fluff and a bottom or a second layer including superabsorbent material in wrapped or unwrapped fluff. Alternatively, the superabsorbent-containing fluff can be located between the two layers. The sanitary napkin 10 may also optionally include a fluid distribution layer 26.

Additionally, FIG. 6 illustrates another embodiment of the fluid intake intensifier pledget 15. One skilled in the art will readily appreciate that such a variation is also applicable to the fluid intake intensifier pledget 15 illustrated in FIG. 2 as well as the fluid intake intensifier pledget 15 illustrated in FIG. 4. The fluid intake intensifier pledget 15 is illustrated in FIG. 6 as including, by way of example only and not by way of limitation, a first layer 16 (illustrated here in an hour-glass configuration) and a second layer 17 (illustrated here in a rectangular configuration). The first layer 16 includes a TABCW material and the second layer 17 includes an airlaid nonwoven material. It should be clear to one skilled in the art, however, that the first layer may be positioned adjacent either the body-facing surface or the garment-facing surface of the second layer.

### EXAMPLE

The following Example illustrates one or more embodiments of the invention.

Other embodiments within the scope of the claims herein will be apparent to one skilled in the art from consideration of the specification or practice of the invention as disclosed herein. It is intended that the specification, together with the Example, be considered exemplary only, with the scope and spirit of the invention being indicated by the claims which follow the Example.

### Example

This Example illustrates tests for a fluid intake intensifier pledget with various amounts of additional absorbent material selected from TABCW material (T), two airlaid materials (A and A′) or combinations thereof, in various further combinations with a cover (C) made of a hydroentangled, hydroapertured spun-lace material.

Specifically, the TABCW material used had a basis weight of about 25 gsm, the airlaid material 1(A) had a basis weight of about 130 gsm and a density of about 0.09 g/cc, and the airlaid material 2 (A′) had a basis weight of about 175 gsm and a density of about 0.08 g/cc. The cover included a homogeneous mixture of about 70% rayon fiber and about 30% PET fiber with a basis weight of about 10 gsm. Each hydroaperture was substantially in a diamond shape made into a mesh pattern by hydroentanglement. The total surface area for the hydroapertures was approximately 25.1%.

A sanitary napkin constructed with a fluid intake intensifier pledget according to the present invention and a cover having the materials identified above, has a cover which provides a cotton-like appearance and feel. For the tests, the following procedures were utilized:

1. Material caliper for thickness measurements: The caliper of a material is a measure of its thickness, and is measured at about 0.05 psi with a Starret-type bulk tester in millimeter (mm) units. In practice, 10 repetitions of any measurement should be made.

2. Rate block intake test: This test was used to determine the intake time of a known quantity of fluid into a material and/or a material system. The test apparatus consisted of a rate block, a funnel and a timer or stop watch. 4 inch×4 inch (102 mm²) pieces of a fluid intake intensifier pledget and a cover were prepared for each test. The cover was placed over the fluid intake intensifier pledget to be tested and the rate block was placed on top of the two materials.

3. Rewet test: This test was used to determine the amount of fluid that will migrate back to the body-facing surface of a cover when a known load is applied. The amount of fluid returning to the surface is called the rewet value. If more fluid comes back to the surface, the rewet value is larger, while smaller amounts of fluid returning to the surface results in lower rewet values. Lower rewet values are associated with a dryer material and hence a dryer feeling sanitary napkin. When considering rewet, three properties are considered significant: (a) intake rate, if the material/system does not have a good intake rate, the fluid has a greater tendency to rewet; (b) fluid retention, the more fluid the absorbent retains, the less fluid is available for rewet; and (c) flow-back, the more fluid prohibited from migrating back through the cover, the lower the rewet.

4. Intake/staining test: An intake/staining test enables observations for stain size, intensity and fluid retention in components to be observed with fluid flow rate and pressure.

For the tests, 2 mL each of a synthetic menstrual fluid formulation known as “Z-Date” was delivered into the test apparatus funnel and the timer was initiated. Z-Date contains, on a weight percent basis, approximately 82.5% water, 15.8% polyvinyl pyrrolidone and 1.7% salts, coloring agents and surfactants. Z-Date, available from PPG Industries, Inc., of Pittsburgh, Pa., has a viscosity of 17 centipoise and a surface tension of 53.5 dynes per centimeter. The Z-Date moved from the funnel into a capillary where it was delivered to the sample. The timer was stopped when all of the Z-Date was substantially absorbed into the sample as observed from the chamber in the test apparatus. The intake time for a known quantity of the Z-Date was recorded for a given sample. This value typically is indicative of a materials system's absorbency, with lower intake time representing a more absorbent system. Five to ten repetitions were performed to determine average intake time.

In the testing, 2 mL of Z-Date was introduced into the rate block apparatus and allowed to absorb into a 4 inch×4 inch sample of the cover material which was placed on top of a 4 inch×4 inch sample of the fluid intake intensifier pledget. The Z-Date was allowed to interact with the sample for approximately 60 seconds with the rate block resting on top of the sample. The sample was placed onto a closed bag partially filled with a saline solution. The fluid bag was positioned on top of a lab jack. Pieces of blotter paper were weighed and placed on top of the sample. The bag with the sample was raised against a fixed acrylic plate using the lab jack until a total of 1 psi was applied.

The pressure was held for approximately 3 minutes after which it was released and the blotter paper weighed. The blotter paper should have retained any Z-Date that was transferred to it from the sample. The difference in weight between the original blotter and the blotter after the absorption experiment was the rewet value.

A sample measuring 4 inches×4 inches was placed beneath an acrylic plate having a ⅛ inch (3 mm) diameter hole bored into the center. A piece of ⅛ inch tubing was connected to the hole with a fitting. Z-Date was delivered to the sample using a syringe pump at a specified rate and for a specified volume. In these experiments, the pump was programmed to deliver a total volume of about 1 mL to the samples which were under pressures ranging between about 0.00 psi and about 0.09 psi. These pressures were applied using a weight that was placed on top of the acrylic plates and evenly distributed. The pump was programmed to deliver at a rate of 1 mL/s.

The stain size area for the tested samples was measured manually and the amount of Z-Date in each component of the system was measured by weight before and after absorption of the Z-Date. Fluid retention was measured by weighing the cover before and after fluid introduction. Average stain size and fluid retention were determined from at least five repetitions at each pressure.

The data illustrated in the following tables were calculated as follows:

CA=[Cover]−[Airlaid 1]

CTA=[Cover]−[TABCW]−[Airlaid 1]

CAA′=[Cover]−[Airlaid 1]−[Airlaid 2]

CTAA′=[Cover]−[TABCW]−[Airlaid 1]−[Airlaid 2]

TABLE 1
Intake

| | Time (s) | Rewet (g) |
|---|---|---|
| CA | 26.29 | 0.85 |
| CTA | 8.06 | 0.80 |
| CAA′ | 9.29 | 0.47 |
| CTAA′ | 5.23 | 0.51 |

TABLE 2
Retention (no load)

| | Retain (g) | Area (mm²) |
|---|---|---|
| CA | 0.28 | 1785 |
| CTA | 0.20 | 1326 |
| CAA′ | 0.17 | 1086 |
| CTAA′ | 0.19 | 1156 |

TABLE 3
Retention (0.09 psi)

| | Retain (g) | Area (mm²) |
|---|---|---|
| CA | 0.39 | 2587 |
| CTA | 0.24 | 1688 |
| CAA′ | Not tested | not tested |
| CTAA′ | 0.21 | 1432 |

As the above results illustrate, the use of a TABCW material in the fluid intake intensifier pledget of the present invention substantially reduces both intake time and rewet. When absorbent capacity is low, use of a fluid intake intensifier pledget of the present invention can help reduce both the intake time and rewet of a sanitary napkin.

In view of the above, it will be seen that the several advantages of the invention are achieved and other advantageous results attained.

As various changes could be made in the above methods and products without departing from the scope of the invention, it is intended that all matter contained in the above description and shown in the accompanying drawings shall be interpreted as illustrative and not in a limiting sense.

What is claimed is:

1. An absorbent article, comprising an absorbent core and an intake intensifier pledget located on a central portion of the absorbent core, the pledget further comprising a material selected from the group consisting of an airlaid nonwoven material, a TABCW material, a composite of a fiber material and an airlaid material, and combinations of a fiber layer and an airlaid layer.

2. The absorbent article of claim 1, wherein the composite has a first layer and a second layer, and wherein the first layer comprises a fiber material and the second layer comprises an airlaid material.

3. The absorbent article of claim 1, wherein the airlaid nonwoven material has a basis weight of between about 50 and about 300 gsm, said basis providing for high void volume.

4. The absorbent article of claim 1, wherein the airlaid nonwoven material includes a superabsorbent material.

5. The absorbent article of claim 1, wherein the TABCW material provides a low densified lofty thru-air bonded carded web and has a basis weight of between about 15 and about 70 gsm.

6. The absorbent article of claim 5, wherein the TABCW material comprises a staple fiber having a denier of between about 3 and about 10.

7. The absorbent article of claim 5, wherein the TABCW material comprises an Ultra Bulky (UB) bicomponent fiber or composites thereof.

8. The absorbent article of claim 1, wherein the pledget further comprises a first layer and a second layer, the first layer comprising a TABCW material and the second layer comprising an airlaid nonwoven material.

9. The absorbent article of claim 1, wherein the pledget comprises a composite of an airlaid nonwoven material and a TABCW.

10. The absorbent article of claim 1, wherein the absorbent core comprises a material selected from the group consisting of a composite of superabsorbent material and pulp, a tissue, a non-woven material and a mixture of fluff and a superabsorbent material.

11. The absorbent article of claim 1, wherein the pledget has a length of at least about 50 mm and a width of from about 30 to about 60 mm.

12. The absorbent article of claim 1, comprising a cover, a wrapping material, and a baffle, wherein the pledget has a first surface situated adjacent the garment-facing surface of the cover and a second surface bonded to at least one of the absorbent core or the wrapping material.

13. The absorbent article of claim 12, further comprising a fluid distribution layer.

14. The absorbent article of claim 13, further comprising an embossed channel having a width of less than about 1 cm and situated adjacent the periphery of the pledget.

15. An absorbent article, comprising a cover, an absorbent core and an intake intensifier pledget located on a central portion of the absorbent core, wherein the cover further comprises a hydroentangled, hydroapertured spun-lace material and the pledget further comprises a TABCW material.

16. The absorbent article of claim 15, wherein the hydroentangled, hydroapertured spun-lace material is rayon fiber.

17. The absorbent article of claim 15, wherein the hydroentangled, hydroapertured spun-lace material is selected from the group consisting of PET polyester, polyethylene, polypropylene and bicomponents thereof.

18. The absorbent article of claim 15, wherein the hydroentangled, hydroapertured spun-lace material is a homogeneous mixture of about 70% rayon fiber and about 30% PET polyester.

19. An absorbent article, comprising a cover, a first absorbent layer and a second absorbent layer, the first absorbent layer being situated between the cover and the second absorbent layer, the cover further comprising a hydroentangled, hydroapertured spun-lace material, the first absorbent layer further comprising a material selected from the group consisting of an airlaid material, a TABCW material and a composite material of a fiber layer and an airlaid layer, and the second absorbent layer further comprising a material selected from the group consisting of an airlaid material, a TABCW material and a composite material of a fiber layer and an airlaid layer.

20. The absorbent article of claim 19, wherein the hydroentangled, hydroapertured spun-lace material is rayon fiber.

21. The absorbent article of claim 19, wherein the hydroentangled, hydroapertured spun-lace material is selected from the group consisting of PET polyester, polyethylene, polypropylene and bicomponents thereof.

22. The absorbent article of claim 19, wherein said a hydroentangled, hydroapertured spun-lace material is a homogeneous mixture of about 70% rayon fiber and about 30% PET polyester.
